(19) United States
(12) Patent Application Publication
(10) Pub. No.: US 2025/0268744 A1
(43) Pub. Date: Aug. 28, 2025

(54) Non-Invasive Female Catheter System

(71) Applicant: Lynette Bassaw, Springfield, OR (US)

(72) Inventor: Lynette Bassaw, Springfield, OR (US)

(21) Appl. No.: 18/585,259

(22) Filed: Feb. 23, 2024

(51) Int. Cl. A61F 5/455; A61F 5/44

(52) U.S. Cl. CPC A61F 5/455; A61F 5/4401; A61F 5/4404

(57) ABSTRACT

A non-invasive female catheter arrangement generally comprises a collection bag, a catheter head, and tubing. The catheter head includes a plastic shell and an absorbent structure mounted on the plastic shell. The plastic shell has an elongated spoon or scoop shape that is concave and substantially wider in the middle portion than at the end portions. The absorbent structure is designed and positioned to fill the plastic shell and form an essentially flat face designed to be positioned to face and or lie on the vulva of a user. The absorbent structure includes a drainage channel positioned therein and connected to the tubing for the drainage of urine of the absorbent structure.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to female catheters and more particularly pertains to a new non-invasive female catheter arrangement for draining urine for persons who are permanently or temporarily bedridden or otherwise unable to utilize a toilet for urination.

### (2) Description of Related Art Including Information Disclosed Under 37 CFR 1.97 and 1.98

The prior art relates to female catheters. The prior art, as best understood, does not disclose a non-invasive female catheter arrangement with a catheter head that has a scoop or spoon shaped outer shell and a gauze layer that fills the outer shell such that the absorbent region is thickest and widest adjacent the urethra of the user.

### BRIEF SUMMARY OF THE INVENTION

An embodiment of the disclosure meets the needs presented above in a non-invasive female catheter arrangement generally comprising a collection bag, a catheter head, and tubing. The collection bag is designed to receive urine. The catheter head is designed to be placed over the vulva of a user. The tubing is designed and positioned to connect the collection bag to the catheter head to conduct urine from the catheter head to the collection bag. The catheter head includes a plastic shell and an absorbent structure mounted on the plastic shell. The plastic shell has an elongated spoon or scoop shape that is concave and substantially wider in the middle portion than at the end portions. The absorbent structure is designed and positioned to fill the plastic shell and form an essentially flat face designed to be positioned to face and or lie on the vulva of a user. The absorbent structure includes a drainage channel positioned therein and connected to the tubing for the drainage of urine of the absorbent structure.

One of the advantages of the above design is that the absorbent region is thickest and widest adjacent the urethra of the user, such that absorbance of urine is maximized. Another advantage is that the catheter does not need to be inserted into the vulva, such as between the labia, which facilitates positioning and use and minimizes irritation to a user. The absorbent material can be selected to conduct moisture away from the surface of the catheter head and into the drain, such that the catheter head remains relatively dry so as to not irritate the skin or other portions of the vulva of the user.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a non-invasive female catheter arrangement according to an embodiment of the disclosure.

FIG. 2 is a top view of a portion of an embodiment of the disclosure.

FIG. 3 is a cross-sectional view of a portion of an embodiment of the disclosure.

FIG. 4 is a top view of an embodiment of the disclosure.

FIG. 5 is a top view of an embodiment of the disclosure in use.

FIG. 6 is a side view of an embodiment of the disclosure in use.

FIG. 7 is a perspective view of another embodiment of the disclosure.

### DETAILED DESCRIPTION OF THE INVENTION

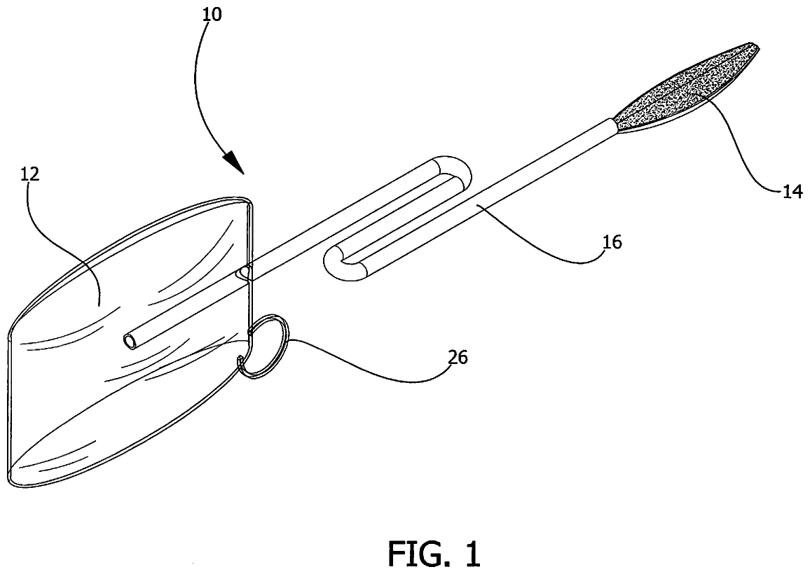

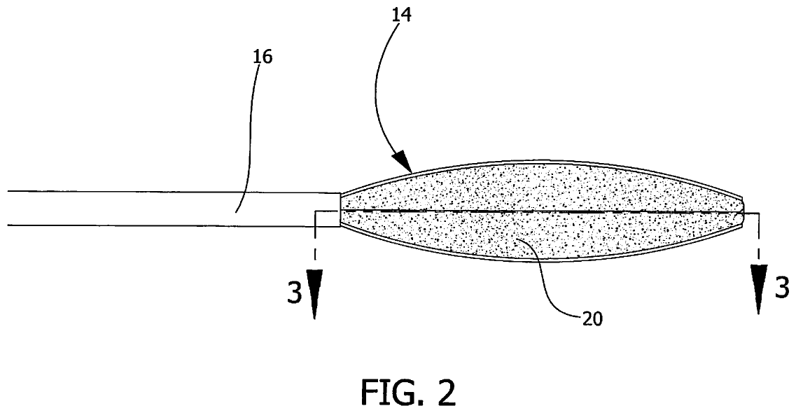

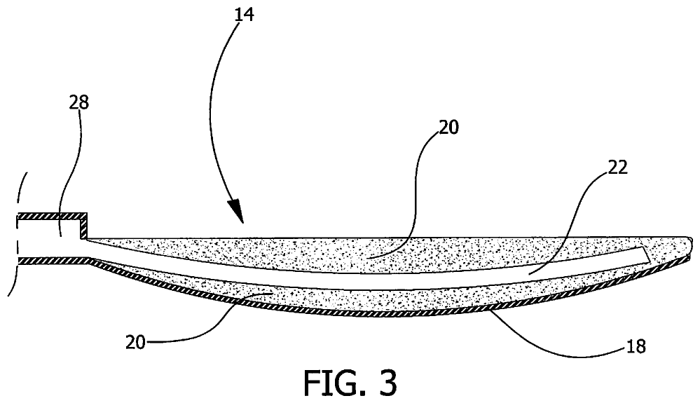

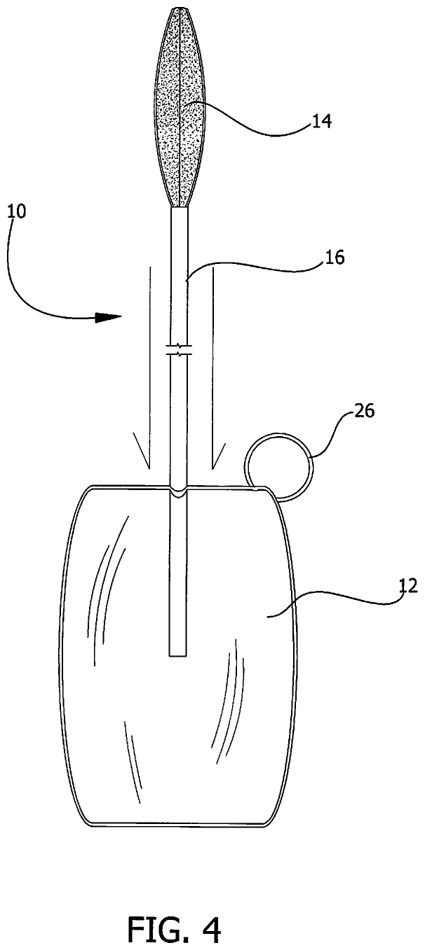

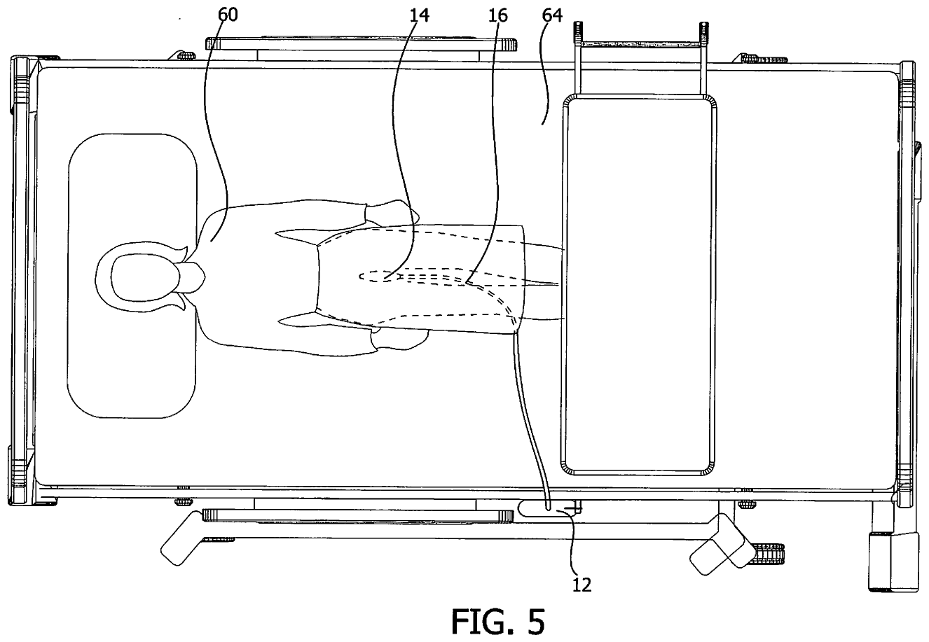

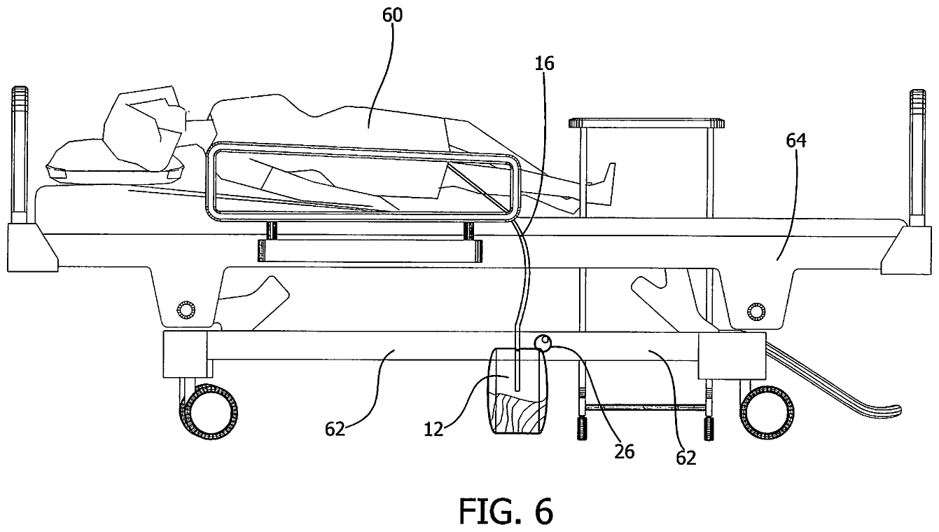

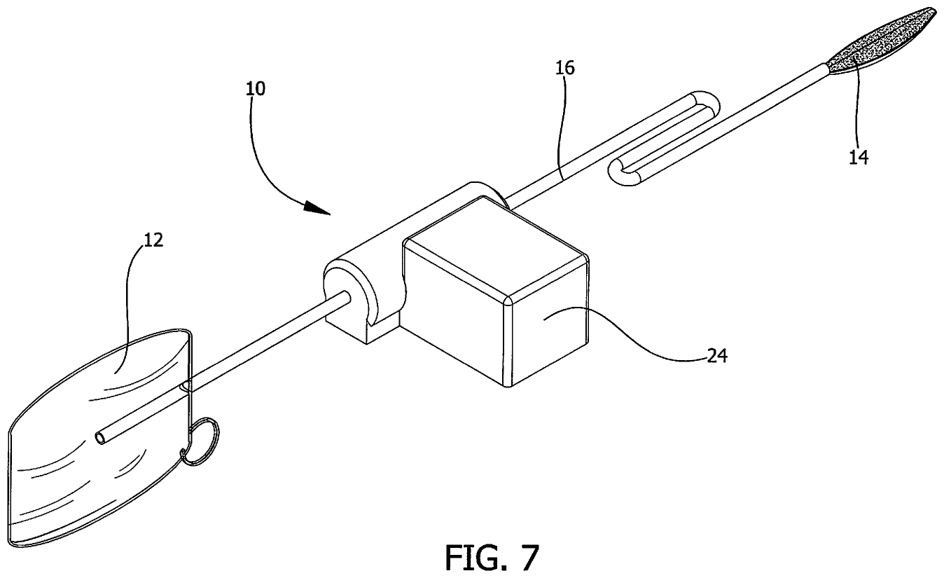

With reference now to the drawings, and in particular to FIGS. 1 through 7 thereof, a new non-invasive female catheter arrangement embodying the principles and concepts of an embodiment of the disclosure and generally designated by the reference numeral 10 will be described.

As best illustrated in FIGS. 1 through 7, the non-invasive female catheter arrangement 10 generally comprises a collection bag 12, a catheter head 14, and tubing 16. The collection bag 12 is designed to receive urine. The catheter head 14 is designed to be placed over the vulva of a user 60. The tubing 16 is designed and positioned to connect the collection bag 12 to the catheter head 14 to conduct urine from the catheter head 14 to the collection bag 12. The catheter head 14 includes a plastic shell 18 and an absorbent structure 20 mounted on the plastic shell 18. The plastic shell 18 has an elongated spoon or scoop shape that is concave and substantially wider in the middle portion than at the end portions. The absorbent structure 20 is designed and positioned to fill the plastic shell 18 and form an essentially flat face designed to be positioned to face and or lie on the vulva of a user 60. The absorbent structure 20 includes a drainage channel 22 positioned therein and connected to the tubing 16 for the drainage of urine of the absorbent structure 20. In accordance with at least one possible embodiment, the plastic shell 18 includes a cylindrical connecting portion 28 configured to be inserted into an open end of the tubing 16.

In accordance with at least one possible embodiment, the absorbent structure 20 includes gauze or similar absorbent material.

In accordance with at least one possible embodiment, the drainage channel 22 has a curved cross-section that essentially follows the concave shape of the plastic shell 18 such that the absorbent structure 20 is thicker in the middle than at the ends to provide maximum absorbent material closest to the urethra of the user 60.

In accordance with at least one possible embodiment, the non-invasive female catheter arrangement 10 includes a pump 24. The pump 24 is positioned between and to connect a first section of the tubing 16 and a second section of the tubing 16. The pump 24 is designed to pump urine through the tubing 16 and into the collection bag 12.

In accordance with at least one possible embodiment, the collection bag 12 includes a hanger 26. The hanger 26 is designed to permit a user 60 to hang the collection bag 12 from a bed frame 62 or similar structure located at a lower height than the catheter head 14 such that urine absorbed by the catheter head 14 drains by gravity into the collection bag 12.

In use, a user 60 places the catheter head 14 over the vulva of the user 60 with the absorbent structure 20 facing or lying on the vulva of the user 60, which user 60 may be bedridden in a hospital bed 64 or otherwise unable to use a toilet for urination. The absorbent structure 20 absorbs expelled urine. Absorbed urine drains through the drainage channel 22, then through the tubing 16, and then into the collection bag 12. In the embodiment using a pump 24, the pump 24 actively pumps urine through the tubing 16 and into the collection bag 12. In the embodiment using gravity, the user 60 hangs the collection bag 12 from a bed frame 62 or similar structure located at a lower height than the catheter head 14 such that urine absorbed by the catheter head 14 drains by gravity into the collection bag 12.

In accordance with at least one possible embodiment, the catheter head 14 is held in place by the underwear of a user 60, though other means of securing the catheter head 14 in place are contemplated by the disclosure.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items 10 following the word are included, but items not specifically mentioned are not excluded. A reference to an element by the indefinite article “a” does not exclude the possibility that more than one of the element is present, unless the context clearly requires that there be only one of the elements.

I claim:

1. A non-invasive female catheter arrangement comprising:
a collection bag being configured to receive urine;
a catheter head being configured to be placed over the vulva of a user;
tubing being configured and disposed to connect said collection bag to said catheter head to conduct urine from said catheter head to said collection bag;
said catheter head comprising a plastic shell and an absorbent structure mounted on said plastic shell;
said plastic shell having an elongated spoon or scoop shape being concave and substantially wider in the middle portion than at the end portions;
said absorbent structure being configured and disposed to fill said plastic shell and form an essentially flat face configured to be disposed to face and or lie on the vulva of a user; and
said absorbent structure comprising a drainage channel disposed therein and connected to said tubing for the drainage of urine of said absorbent structure.

2. The non-invasive female catheter arrangement of claim 1, wherein said absorbent structure comprises gauze or similar absorbent material.

3. The non-invasive female catheter arrangement of claim 2, wherein said drainage channel has a curved cross-section that essentially follows the concave shape of said plastic shell such that said absorbent structure is thicker in the middle than at the ends to provide maximum absorbent material closest to the urethra of the user.

4. The non-invasive female catheter arrangement of claim 3, wherein:
said non-invasive female catheter arrangement comprises a pump;
said pump is disposed between and to connect a first section of said tubing and a second section of said tubing; and
said pump is configured to pump urine through said tubing and into said collection bag.

5. The non-invasive female catheter arrangement of claim 3, wherein:
said collection bag comprises a hanger; and
said hanger is configured to permit a user to hang said collection bag from a bed frame or similar structure located at a lower height than said catheter head such that urine absorbed by said catheter head drains by gravity into said collection bag.

6. The non-invasive female catheter arrangement of claim 1, wherein said drainage channel has a curved cross-section that essentially follows the concave shape of said plastic shell such that said absorbent structure is thicker in the middle than at the ends to provide maximum absorbent material closest to the urethra of the user.

7. The non-invasive female catheter arrangement of claim 1, wherein:
said non-invasive female catheter arrangement comprises a pump;
said pump is disposed between and to connect a first section of said tubing and a second section of said tubing; and
said pump is configured to pump urine through said tubing and into said collection bag.

8. The non-invasive female catheter arrangement of claim 1, wherein:
said collection bag comprises a hanger; and
said hanger is configured to permit a user to hang said collection bag from a bed frame or similar structure located at a lower height than said catheter head such that urine absorbed by said catheter head drains by gravity into said collection bag.

9. A method of using the non-invasive female catheter arrangement of claim 1, said method comprising the steps of:
placing said catheter head over the vulva of a user with said absorbent structure facing or lying on the vulva of a user;
absorbing expelled urine into said absorbent structure; and
draining absorbed urine through said drainage channel, then through said tubing, and then into said collection bag.

10. The non-invasive female catheter arrangement of claim 9, wherein:
said non-invasive female catheter arrangement comprises a pump;
said pump is disposed between and to connect a first section of said tubing and a second section of said tubing; and
said method further comprises pumping urine with said pump through said tubing and into said collection bag.

11. The non-invasive female catheter arrangement of claim 9, wherein:
said collection bag comprises a hanger; and
said method further comprises hanging said collection bag with said hanger from a bed frame or similar structure located at a lower height than said catheter head such that urine absorbed by said catheter head drains by gravity into said collection bag.
